(19) United States
(12) Patent Application Publication
(10) Pub. No.: US 2026/0232886 A1
(43) Pub. Date: Aug. 13, 2026

(54) Control Method and System for Extracorporeal Membrane Oxygenation

(71) Applicant: Université Libre De Bruxelles, Brussels (BE)

(72) Inventors: Chloé Goemans, Molenbeek-Saint-Jean (BE); Nicolas Baeyens, Auderghem (BE); Frédéric Vanden Eynden, Lillois (BE)

(21) Appl. No.: 19/160,718

(22) PCT Filed: Mar. 1, 2024
(86) PCT No.: PCT/EP2024/055311

§ 371 Date: Aug. 28, 2025

(30) Foreign Application Priority Data
Mar. 1, 2023 (EP) 23159316.1

(51) Int. Cl. A61M 1/36

(52) U.S. Cl. CPC A61M 1/3603; A61M 1/3607; A61M 2202/0007; A61M 2202/0021; A61M 2202/0208; A61M 2202/0413; A61M 2205/3327; A61M 2205/3334; A61M 2205/50; A61M 2230/005; A61M 2230/20

(57) ABSTRACT

Presently described is a method and system for dynamically determining a target flow rate of a pump of a system for providing veno-arterial extracorporeal membrane oxygenation, VA-ECMO, of a subject during a VA-ECMO session comprising receiving multiple sets of measured parameters of the subject, wherein each set of the multiple sets of measured parameters comprises heart contractility indicator, and the multiple sets of measured parameters are received over time during the VA-ECMO session, determining, using the multiple sets of measured parameters, the target flow rate of the pump, wherein in response to a stable or increase in heart contractility indicator, the target flow rate is adjusted by a reduction, and in response to a decrease in heart contractility indicator, the target flow rate is adjusted by an increase. Also described is a device for interfacing to a VA-ECMO system, device is configured for carrying out the method.

### CROSS REFERENCE TO RELATED APPLICATIONS

This application is a national phase entry under 35 U.S.C. § 371 of International Patent Application PCT/EP2024/055311, filed Mar. 1, 2024, designating the United States of America and published in English as International Patent Publication WO 2024/180202 on Sep. 6, 2024, which claims the benefit under Article 8 of the Patent Cooperation Treaty to European Patent Application Serial No. 23159316.1, filed Mar. 1, 2023, the entireties of which are hereby incorporated by reference.

### FIELD OF THE INVENTION

The present invention is in a field of veno-arterial configured extracorporeal membrane oxygenation (VA-ECMO), in particular, a method and system for controlling flow rate thereof. Background to the invention

When used in its veno-arterial configuration, extracorporeal membrane oxygenation (VA-ECMO) provides both a cardiac and a pulmonary support to the patient. VA-ECMO removes deoxygenated blood from a patient vein, usually a right femoral vein, artificially oxygenates it, and pumps it back into a patient artery, usually the left femoral artery.

This VA-ECMO can be a lifesaving procedure for the patient with severe heart or lung dysfunction, who are unable to receive enough oxygen through normal circulation. Examples of dysfunctions include acute cardiac infarction, a decompensated heart failure or a fulminant myocarditis. It can also be used after cardiac surgery for patients unable to be weaned from the cardiopulmonary bypass or for the transplanted patient with a primary graft dysfunction.

Despite its wide scope and potential life-saving nature, the outcome of the use of VA-ECMO is still affected by a high mortality during the procedure. The overall survival rate for VA-ECMO use in adult patients is approximated to 44%. More concerning, the survival rate at 24 month is only slightly higher than 60% of the patients who survived the procedure.

A significant drawback of VA-ECMO is that an afterload of the left ventricle is increased by an antegrade flow of the mechanical support. For instance, when set to its full flow, the VA-ECMO will cause a high mean arterial pressure. With the mean arterial pressure elevated, pressure generated by contraction of the left ventricle will need to increase correspondingly in order to open the aortic valve. However, patients suffering from cardiac pathologies have a decreased inotropy, meaning that they cannot generate the required pressure. As a result, the aortic valve will not be able to open, pressure builds up in the left ventricle, its volume increases to accommodate the increased pressure. This causes an increase in the cardiac wall tension, which creates stress. Furthermore, it prevents the heart from being perfused optimally, leading to toxic inflammatory injuries (A. Solholm et. al., Myocardial perfusion and cardiac dimensions during extracorporeal membrane oxygenation-supported circulation in a porcine model of critical post-cardiotomy failure, 2020, Perfusion).

A further drawback of VA-ECMO is a risk of the patient developing Harlequin (or North-South) syndrome. This phenomenon occurs when the aortic valve is able to open, but blood discharged from the left ventricle is poorly oxygenated due to the patient pathology. In VA-ECMO, the inflow cannula carrying artificially oxygenated blood is generally located in the femoral artery, meaning there is some distance between the outflow of the left ventricle (poorly oxygenated blood) and the outflow of the VA-ECMO cannula (artificially oxygenated blood). The respective blood outflows converge in a mixing cloud whose distance from the heart is proportional to the force of the heart's remaining contractions and inversely proportional to the VA-ECMO flow rate. In cases where said distance is located after (inferior to) the branching of the subclavian artery, the brain is irrigated with deoxygenated blood, leading to life-threatening cerebral hypoxemia.

A further drawback of VA-ECMO is its weaning process. Reducing the flow of the VA-ECMO is typically a manual operation, in which the health care specialist manually reduces blood flow. In a typical weaning process of the art, the patient's arterial pressure (AP) is measured with the help of a pressure catheter. At the start of the VA-ECMO session the AP signal is usually constant or have a very low pulsatility. During this period the VA-ECMO flow rate is kept at its maximum value. When the patient has regained a sufficient pulsatility in their AP signal, there is a possibility that the heart is able to take over from the pump. The medical personal will reduce the flow of the VA-ECMO to a just above zero (0.5 L/min prevent the blood from clotting). An echocardiological examination is subsequently to measure velocity-time integral (VTI). From this measurement, the cardiac output of the patient can be estimated. If it is sufficient, the ECMO is weaned. If it is not, the ECMO is put back at its high flow rate. This process is long, inefficient and causes a high workload on the, already overworked, medical staff.

A further drawback of VA-ECMO is a gradual loss of heart contractility during the VA-ECMO session. As mentioned earlier, a full-flow VA-ECMO prevents the heart from ejecting blood, so preventing the heart from beating properly. As a result, cardiac muscle slowly loses its capacity to contract, similar to any other muscle tissue that is not used for a long period of time.

### SUMMARY OF THE INVENTION

Presently described is a method for dynamically determining a target flow rate of a pump of a system for providing veno-arterial extracorporeal membrane oxygenation, VA-ECMO, of a subject during a VA-ECMO session comprising:
receiving multiple sets of measured parameters of the subject, wherein:
   each set of the multiple sets of measured parameters comprises heart contractility indicator, and
   the multiple sets of measured parameters are received over time during the VA-ECMO session,
determining, using the multiple sets of measured parameters, the target flow rate of the pump, wherein:
   in response to a stable or increase in heart contractility indicator, the target flow rate is adjusted by a reduction, and
   in response to a decrease in heart contractility indicator, the target flow rate is adjusted by an increase.

Further provided is a computer-implemented method for dynamically determining a target flow rate of a pump of a system for providing veno-arterial extracorporeal membrane oxygenation, VA-ECMO, of a subject during a VA-ECMO session comprising:
receiving multiple sets of measured parameters of the subject, wherein:
   each set of the multiple sets of measured parameters comprises heart contractility indicator; and
   the multiple sets of measured parameters are received over time during the VA-ECMO session;
determining, using the multiple sets of measured parameters, the target flow rate of the pump, wherein:
   in response to a stable or increase in heart contractility indicator, a value of the target flow rate is adjusted by a reduction; and
   in response to a decrease in heart contractility indicator, a value of the target flow rate is adjusted by an increase;
output the target flow rate of the pump.

Further provided is a device for interfacing to a system for providing veno-arterial extracorporeal membrane oxygenation VA-ECMO, a VA-ECMO system, to a subject during a VA-ECMO session, wherein the device is configured for dynamically determining a target flow rate of a pump of a the VA-ECMO system for providing veno-arterial extracorporeal membrane oxygenation, VA-ECMO, of a subject during a VA-ECMO session using a method comprising:
receiving multiple sets of measured parameters (120) of the subject, wherein:
each set (120) of the multiple sets of measured parameters comprises heart contractility indicator, and
the multiple sets of measured parameters are received over time during the VA-ECMO session,
determining (140), using the multiple sets of measured parameters, the target flow rate of the pump (160), wherein:
in response to a stable or increase in heart contractility indicator, the target flow rate is adjusted by a reduction, and
in response to a decrease in heart contractility indicator, the target flow rate is adjusted by an increase.

Preferably, each set of the multiple sets of measured parameters further comprises an end-organ perfusion indicator, wherein the target flow rate of the pump is adjusted by an increase when the measured end-organ perfusion indicator is outside an accepted range.

Preferably, each set of the multiple sets of measured parameters further comprises an oxygen saturation indicator, wherein the target flow rate of the pump is adjusted by an increase when the measured oxygen saturation indicator is less than a threshold value.

Preferably, the target flow rate of the pump is received by a feedback loop controller wherein the feedback loop controller is configured to:
receive a measured flow rate of the pump, that is a measurement of flow rate of the pump,
output a flow rate setting for the pump such that the measured flow rate of the pump approaches the determined flow rate of the pump.

Preferably, each set of the multiple sets of measured parameters comprises:
the heart contractility indicator as described herein,
the end-organ perfusion indicator as described herein, and
the oxygen saturation indicator as described herein,
   wherein
   if
the oxygen perfusion indicator is below the threshold value, or
the venous oxygen saturation indicator is below the threshold value, or
there is a decrease in the heart contractility indicator
   then
the determined target flow rate of the pump is adjusted by an increase
   else
in response to a stable or increase in heart contractility indicator, the determined target flow rate is adjusted by a decrease.

Preferably, each set of the multiple sets of measured parameters comprises:
the heart contractility indicator as described herein,
the end-organ perfusion indicator as described herein, and
the oxygen saturation indicator as described herein,
   wherein
   a) if the end-organ perfusion indicator is outside the accepted range,
   then
the target flow rate of the pump is adjusted by an increase, and there is a return to step a);
   else
   b) if the oxygen saturation indicator is below the threshold value,
   then
the target flow rate of the pump is adjusted by an increase, and there is a return to step a);
   else
   c) if there is a decrease in the heart contractility indicator,
   then
the target flow rate of the pump is adjusted by an increase, and there is a return to step a);
   else
   the target flow rate of the pump is adjusted by a decease, and there is a return to step a);
   wherein
end-organ perfusion indicator of step a) is determined for a duration EOPI_{sampleperiod},
the oxygen saturation indicator of step b) is determined for a duration OSI_{sampleperiod},
there are multiple EOPI_{sampleperiod} running consecutively and there are multiple OSI_{sampleperiod} running consecutively, each EOPI_{sampleperiod} and OSI_{sampleperiod} are of the same length, start at the same time and run concurrently,
the heart contractility indicator of step c) is determined for a duration HCI_{sampleperiod},
there are multiple HCI_{sampleperiods} in a session running consecutively,
during each HCI_{sampleperiod}, the multiple consecutively-running EOPI_{sampleperiods} run currently with the HCI_{sampleperiod},
during the HCI_{sampleperiod}, the multiple consecutively-running OSI_{sampleperiods} run currently with the HCI_{sampleperiod},
the target flow rate increase of step a) is implemented for a duration I_{period}, which starts after each EOPI_{sampleperiod} and runs concurrently with a subsequent EOPI_{sampleperiod}, or the target flow rate increase of step b) is implemented for the duration I_{period}, which starts after each OSI_{sampleperiod}, and runs concurrently with a subsequent OSI_{sampleperiod}, or the target flow rate increase or decrease of step c) is implemented for the duration I_{period}, which starts after each HCI_{sampleperiod}, and runs concurrently with a part of the subsequent HCI_{sampleperiod},
a duration of the I_{period} is the same or less than a duration of the EOPI_{sampleperiod} or OSI_{sampleperiod}.

The flow rate of the pump is preferably adjusted to the target flow rate using a device (interface device) for interfacing with the VA-ECMO system, an interface device, configured to adjust the flow rate of the pump responsive to determined target flow rate.

Preferably, the method is a computer implemented method.

Further provided is a device (interface device) for interfacing to a system for providing veno-arterial extracorporeal membrane oxygenation VA-ECMO of a subject during a VA-ECMO session, wherein
the device is configured to dynamically determining the flow rate of a pump of the system according to the method as described herein.

The interface device may further comprise a dismountable actuator assembly (320) for interfacing to the VA-ECMO system (200), the dismountable actuator assembly (320) configured to:
dismountably attach to a manually-actuated control dial (220) of the VA-ECMO system (200), wherein the manually-actuated control dial (220) adjusts the flow rate of the pump of the VA-ECMO system (200); and
adjust by motorized actuation of the control dial (220) the flow rate of the pump of the VA-ECMO system (200), responsive to the determined target flow rate of the pump.

The device may be at least partially comprised in an external module configured for control of the flow rate of the pump of the VA-EMCO system, wherein:
the external module comprises a control signal output port for connection to a control signal input port of the VA-EMCO system, and
signals outputted by the control signal output port control of the flow rate of the pump of the VA-EMCO system.

The device may be comprised in a circuit board configured for electrical connection to existing circuitry of the VA-ECMO and for control of the flow rate of the pump thereof.

Further provided is a system for providing veno-arterial extracorporeal membrane oxygenation VA-ECMO of a subject during a VA-ECMO session, wherein
the system is at least partially configured to dynamically determine the flow rate of a pump of the system according to the method as described herein.

Further provided is a computing device or system configured for performing the method as described herein.

Further provided is a computer program or computer program product having instructions which when executed by a computing device or system cause the computing device or system to perform the method as described herein.

Further provided is a computer readable medium having stored thereon a computer program having instructions which when executed by a computing device or system cause the computing device or system to perform the method as described herein.

Further provided is a data stream which is representative of a computer program or computer program product having instructions which when executed by a computing device or system cause the computing device or system to perform the method as described herein.

### FIGURE LEGENDS

FIG. 1: A schematic illustration of a method as described herein.

FIG. 2 is a schematic illustration of a plurality of consecutive HCI_{sampleperiods}, a plurality of I periods each running concurrently with a subsequent HCI_{sampleperiod}.

FIG. 2A is an exemplary flow chart for determining target flow rate based on heart contractility indicator (HCI) at each point “A” in FIG. 2.

FIG. 3 is a schematic illustration of a HCI_{sampleperiod}, a plurality of consecutive EOPI_{sampleperiods} running within the HCI_{sampleperiod}, and a plurality of I_{periods} each running concurrently with a subsequent EOPI_{sampleperiod}.

FIG. 3A is an exemplary flow chart for determining target flow rate based on end organ perfusion indicator (EOPI) at each point “B” in FIG. 3.

FIG. 3B is an exemplary flow chart for determining target flow rate based on end organ perfusion indicator (EOPI) and heart contractility indicator (HCI) at each point “C” in FIG. 3.

FIG. 4 is a schematic illustration of a HCI_{sampleperiod}, a plurality of consecutive OSI_{sampleperiods} running within the HCI_{sampleperiod}, and a plurality of I_{periods} each running concurrently with a subsequent OSI_{sampleperiod}.

FIG. 4A is an exemplary flow chart for determining target flow rate based on oxygen saturation indicator (OSI) at each point “D” in FIG. 4.

FIG. 4B is an exemplary flow chart for determining target flow rate based on oxygen saturation indicator (OSI) and heart contractility indicator (HCI) at each point “E” in FIG. 4.

FIG. 5 is a schematic illustration of a HCI_{sampleperiod}, a plurality of consecutive EOPI_{sampleperiods} and a plurality of consecutive OSI_{sampleperiods} running within the HCI_{sampleperiod}, and a plurality of I_{periods} each running concurrently with a subsequent OSI_{sampleperiod}/EOPI_{sampleperiod}.

FIG. 5A is an exemplary flow chart for determining target flow rate based on end organ perfusion indicator (EOPI) and oxygen saturation indicator (OSI) at each point “F” in FIG. 5.

FIG. 5B is an exemplary flow chart for determining target flow rate based on end organ perfusion indicator (EOPI), oxygen saturation indicator (OSI) and heart contractility indicator (HCI) at each point “G” in FIG. 5.

FIG. 6: Graph showing readings from flow rate, heart contractility indicator (dP/dtₘₐₓ), end-organ perfusion indicator (MAP), and oxygen saturation indicator (SvO₂) of a subject in cardiovascular shock supported by a VA-ECMO controlled by the present method; time is 2.5 to 5.4 hours after initiation of a cardiovascular shock.

FIG. 7: Continuation of graph of FIG. 6, duration is ~5 to 9.5 hours after initiation of a cardiovascular shock.

FIG. 8: Continuation of graph of FIG. 7 duration is ~9 to 12.5 hours after initiation of a cardiovascular shock.

FIG. 9: Graph showing readings from flow rate, heart contractility indicator (dP/dtₘₐₓ), end-organ perfusion indicator (MAP), and oxygen saturation indicator (SvO₂) of a healthy subject supported by a VA-ECMO controlled by the present method.

FIG. 10: Basic illustration of an exterior of a VA-ECMO system of the art and some features thereof including a manually-actuated control dial and pump.

FIG. 11: Basic illustration of an interface device comprising a dismountable actuator assembly (320).

FIG. 12: Basic illustration of the interface device of FIG. 11 interfaced to the manually-actuated control dial of the VA-ECMO system of FIG. 10.

### DETAILED DESCRIPTION OF INVENTION

Before the present system and method of the invention are described, it is to be understood that this invention is not limited to particular systems and methods or combinations described, since such systems and methods and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, >4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the present description of the invention, reference is made to the accompanying drawings that form a part hereof, and in which are shown by way of illustration only of specific embodiments in which the invention may be practiced. Parenthesized or emboldened reference numerals affixed to respective elements merely exemplify the elements by way of example, with which it is not intended to limit the respective elements. Unless otherwise indicated, all figures and drawings in this document are not to scale and are chosen for the purpose of illustrating different embodiments of the invention. In particular the dimensions of the various components are depicted in illustrative terms only, and no relationship between the dimensions of the various components should be inferred from the drawings, unless so indicated.

It is to be understood that other embodiments may be utilised and structural or logical changes may be made without departing from the scope of the present invention. The following detailed description, therefore, is not to be taken in a limiting sense, and the scope of the present invention is defined by the appended claims.

Provided herein is a method for dynamically determining a target flow rate of a pump of a system for providing veno-arterial extracorporeal membrane oxygenation, VA-ECMO, of a subject during a VA-ECMO session.

The method comprises receiving multiple sets of measured parameters of the subject over time during the VA-ECMO session. Each set of the multiple sets of measured parameters comprises at least a heart contractility indicator. The target flow rate of the pump is determined using the multiple sets of measured parameter. In response to a stable or increase in heart contractility indicator, the target flow rate is adjusted by a reduction. In response to a decrease in heart contractility indicator, the target flow rate is adjusted by an increase.

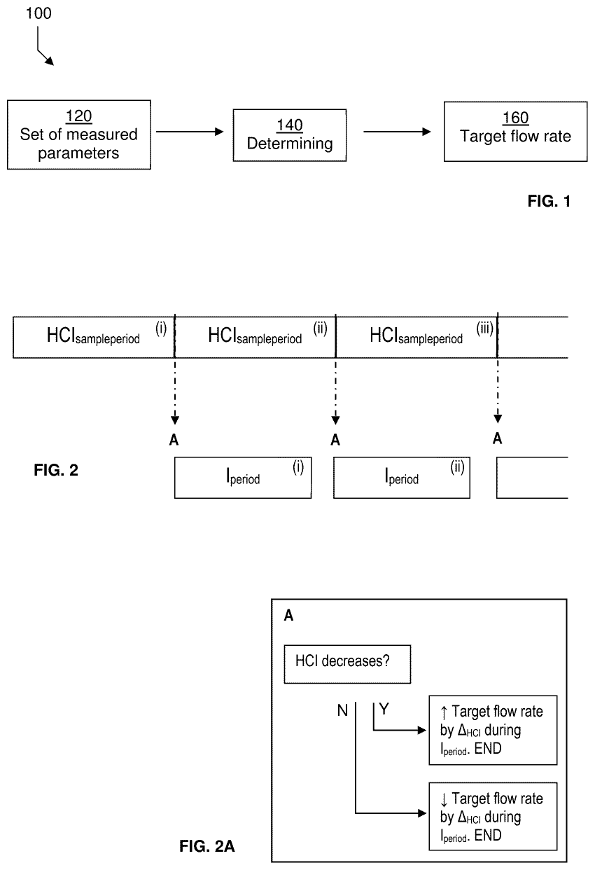

The method (100) is schematically set out in FIG. 1. In FIG. 1, a general scheme is set out wherein a set of measured parameters (120) is received and a target flow rate (160) is determined (140) for the set of measured parameters (120). The target flow rate (160) is determined (140) dynamically from the set of measured parameters (120) acquired at different times.

By dynamically, it is meant that target flow rate of a pump is determined at a plurality of different time points during the session, from a set of measured parameters acquired from the subject at the plurality of different (earlier) time points during the session. By dynamically determining the target flow rate of the pump, the target flow rate responds to changes in the set of measured parameters. If the set of measured parameters evolves or changes over time, there is a corresponding evolution or change of the target flow rate over time. A dynamic implementation typically involves sampling the set of measured parameters at intervals (e.g. every 0.5 to 10 seconds), and determining a target flow rate of a pump for each sampled set of measured parameters or for a sample period containing multiple sets of measured parameters. Dynamically may also be known as “in real-time” or “moment-to-moment”.

A “VA-ECMO session” is a treatment using VA-ECMO of a patient in need thereof. A VA-ECMO session is usually applied in a treatment of patient with severe heart or lung dysfunction, who are unable to receive enough oxygen through normal circulation. Examples of dysfunctions include acute cardiac infarction, decompensated heart failure, fulminant myocarditis, cardiac surgery patients unable to be weaned from the cardiopulmonary bypass, or for the transplanted patient with a primary graft dysfunction. A VA-ECMO typical session typically lasts continuously for 24 hours to several days or weeks.

A subject is typically a human subject (e.g. adult, child, toddler, infant, neonate). However, any mammal is foreseen including, for instance, dog, cat, horse, cattle, sheep, goat, pig, ferret, rabbit, non-human primates, mice and rat.

The present method allows an automated weaning-off the VA-ECMO. The measurement of heart contractility and dynamic determining of the target flow rate allow a gradual decrease over time in flow contribution by the VA-ECMO. The decrease is caused by the method that will lower the target flow rate when the heart contractility is stable.

An indication of status for weaning-off the VA-ECMO is provided by the dynamically controlled flow rate of pump. When the target flow rate stabilised within a threshold range (e.g. less than 0.5 to 1 L/min), it is indicative that the heart is able to take over the circulation provided by the VA-ECMO, and the subject can be removed from the VA-ECMO.

Heart contractility is known in the art, and is also termed in the art as cardiac contractility, myocardial contractility, or inotropy. Heart contractility is the ability of the heart to eject blood at a given arterial pressure and end-diastolic volume. It refers to a force of contraction of the heart muscle. An increase in heart contractility is indicative of an increase in force of contraction and a higher volume of blood ejected in one contraction. Heart contractility indicator is measurement made of a subject that is a heart contractility or that is correlated to heart contractility. The heart contractility indicator has a value that changes in proportion to changes in heart contractility of the subject.

Heart contractility indicator may be measured by any method known in the art. Examples include:
dP/dtₘₐₓ measurement. dP/dtₘₐₓ is the maximal rate of rise of (usually) left ventricular pressure (LVP). It is typically measured using baroinometry or pressure measurement in the left ventricle or an artery. An exemplary method for determining dP/dtₘₐₓ is disclosed in Monge Garcia, M. I. et al. Performance comparison of ventricular and arterial dP/dtₘₐₓ for assessing left ventricular systolic function during different experimental loading and contractile conditions. Crit. Care Lond. Engl. 22, 325 (2018).
end-systolic pressure-volume relationship (ESPVR) measurement. ESPVR is a maximal pressure that can be developed by the ventricle at a given left ventricular volume. It is typically measured using a pressure-volume catheter located in the left ventricle. An exemplary method for determining ESPVR is disclosed in Bastos, M. B. et al. Invasive left ventricle pressure-volume analysis: overview and practical clinical implications. Eur. Heart J. 41, 1286-1297 (2020).
end systolic elastance (Ees) measurement. Ees is derived from a pressure-volume (P-V) loop of the subject which plots changes in ventricular pressure associated with the changes in volume which occur during the cardiac cycle; the Ees is a slope of a line through an end-systolic pressure-volume point (the left upper corner of the P-V loop). A P-V loop is typically measured using a pressure-volume catheter located in the left ventricle. An exemplary method for determining Ees is disclosed in Bastos, M. B. et al. Invasive left ventricle pressure-volume analysis: overview and practical clinical implications. Eur. Heart J. 41, 1286-1297 (2020).
Ejection fraction measurement: Ejection fraction is a measurement of the percentage of blood leaving the heart each time it contracts compared to the volume of blood present in the heart. It is typically measured using doppler echocardiography (external). An exemplary method for determining ejection fraction is disclosed in Liu, D., Peck, I., Dangi, S., Schwarz, K. Q. & Linte, C. A. Left Ventricular Ejection Fraction Assessment: Unraveling the Bias between Area- and Volume-based Estimates. Proc. SPIE—Int. Soc. Opt. Eng. 10955, 109550T (2019).
Shortening fraction (SF) measurement: Shortening fraction (SF) is a measurement of a percentage change in left ventricular diameter during systole. It is typically measured using doppler echocardiography (external). End-systolic and end-diastolic left ventricular diameters are measured and compared. An exemplary method for determining shortening fraction (SF) is disclosed in Rao, P. S. Echocardiography: an overview-part I. Vessel Plus 6, 24 (2022).
Stroke volume measurement: Stroke volume is a difference between end-diastolic and end-systolic volumes; it is the volume ejected with each heart beat. It is typically measured using pressure-volume catheter located in the left ventricle or using doppler echocardiography (external). An exemplary method for determining stroke volume is disclosed in Rao, P. S. Echocardiography: an overview-part I. Vessel Plus 6, 24 (2022).

As mentioned, in response to a stable or increase in heart contractility indicator, the target flow rate is adjusted by a reduction. In response to a decrease in heart contractility indicator, the target flow rate is adjusted by an increase.

To determine the reduction or increase, the heart contractility indicator is typically measured for a heart contractility indicator sample period (HCI_{sampleperiod}). The HCIS_{period} is typically between 1 and 10 minutes, preferably between 2 and 5 minutes. Consecutive HCI_{sampleperiods} preferably run successively, i.e. at the end of a first HCI_{sampleperiod}, a second HCI_{sampleperiod} starts. Within a session, the duration of each HCI_{sampleperiod} is preferably the same.

A value of heart contractility indicator is preferably determined for the HCI_{sampleperiod}. The heart contractility indicator for the HCI_{sampleperiod} is typically a statistical indicator for heart contractility indicator values acquired during the HCI_{sampleperiod} such as, for instance, an order statistic (1st quartile, median, 3rd quartile) or an average (algebric, geometric). Preferably it is a median.

An increase in heart contractility indicator is preferably determined as an increase in heart contractility indicator for a HCI_{sampleperiod} pair. The HCI_{sampleperiod} pair comprises a current HCI_{sampleperiod} and a previous HCI_{sampleperiod}. The previous HCI_{sampleperiod} is preferably the HCI_{sampleperiod} immediately prior to the current HCI_{sampleperiod} (1x earlier), or is the HCI_{sampleperiod} n times earlier to the current HCI_{sampleperiod} In may be decided by the user before the session starts). Where there is an increase, the HCI in a current HCI_{sampleperiod} is greater than the HCI in a previous HCI_{sampleperiod}. The increase for the HCI_{sampleperiod} pair is preferably greater than or equal to 10%.

A decrease in heart contractility indicator is preferably determined as a decrease in heart contractility indicator for a HCI_{sampleperiod} pair. The HCI_{sampleperiod} pair comprises a current HCI_{sampleperiod} and a previous HCI_{sampleperiod}. The previous HCI_{sampleperiod} is preferably the HCI_{sampleperiod} immediately prior to the current HCI_{sampleperiod} (1x earlier), or is the HCI_{sampleperiod} n times earlier to the current HCI_{sampleperiod}. (n may be decided by the user before the session starts). Where there is an decrease, the HCI in a current HCI_{sampleperiod} is lower than the HCI in a previous HCI_{sampleperiod}. The decrease for the HCI_{sampleperiod} pair is preferably greater than or equal to 10%.

A stable heart contractility indicator is preferably determined as no effective change in heart contractility indicator for a HCI_{sampleperiod} pair. The HCI_{sampleperiod} pair comprises a current HCI_{sampleperiod} and a previous HCI_{sampleperiod}. The previous HCI_{sampleperiod} is preferably the HCI_{sampleperiod} immediately prior to the current HCI_{sampleperiod} (1x earlier), or is the HCI_{sampleperiod} n times earlier to the current HCI_{sampleperiod}. In may be decided by the user before the session starts). Where there is no effective change, the HCI in a current HCI_{sampleperiod} is less than 10% different from the HCI in a previous HCI_{sampleperiod}.

The increase or decrease in target flow rate is implemented during an implementation period (I_{period}). During the implementation period (I_{period}), the target flow rate is gradually increased or decreased. The implementation period (I_{period}) is typically between 1 to 100 seconds, preferably between 1 to 10 seconds, preferably between 1 to 5 seconds.

The increase in target flow rate during the implementation period (I_{period}) is typically 0.05-0.5 L/min, more preferably 0.1-0.25 L/min. The decrease in target flow rate during the implementation period (I_{period}) is typically 0.05-0.5 L/min, more preferably 0.1-0.25 L/min. The change in target flow rate due to heart contractility indicator may be termed Δ_{HCI}.

Once the implementation period (I_{period}) has expired, the target flow rate is maintained at the same level as at the end of the implementation period (I_{period}).

Preferably the multiple HCI_{sampleperiods} within a session run consecutively. For instance, after the end of an HCI_{sampleperiod} a subsequent HCI_{sampleperiod} starts. Consecutive HCI_{sampleperiods} ((i), (ii), (iii)) are illustrated in FIG. 2.

Preferably, at the end of the HCI_{sampleperiod}, an adjustment is made to the target flow rate. The adjustment is implemented during the implementation period (I_{period}). The I_{period} preferably runs subsequent to the previous HCI_{sampleperiod} and runs concurrently with the subsequent HCI_{sampleperiod}. For instance, the beginning of I_{period} may start after the end of the previous HCI_{sampleperiod}. For instance, the beginning of I_{period} may start at the same time as the beginning of the subsequent HCI_{sampleperiod}. A duration of each I_{period} may be the same (e.g. between 1 and 10 seconds, preferably between 1 and 5 seconds). Arrangement of HCI_{sampleperiods} (i), (ii), (iii) and subsequent I_{periods} (i), (ii) is shown in FIG. 2. Based on a change in value of HCI between different HCI_{sampleperiods} at each point “A”, the target flow is increased or decreased (FIG. 2A).

Heart contractility indicator may be measured at intervals during the HCI_{sampleperiod} e.g. every 1 to 5 seconds.

Each set of the multiple sets of measured parameters may further comprise an end-organ perfusion indicator, wherein the target flow rate of the pump is further adjusted by an increase when the measured end-organ perfusion indicator is outside an accepted range.

By monitoring and reacting to end-organ perfusion indicator, the present method prevents or reduces organ damage caused by too low oxygen saturation (end organ ischemia) or a lack of the necessary nutriments. The method determines the target flow rate based on heart contractility within safety limits i.e. within the accepted threshold range. By combining the monitoring of and reacting to end-organ perfusion indicator with monitoring of and reacting to heart contractility indicator, the patient is slowly weaned off the VA-ECMO during the session, while reducing risk of end organ ischemia.

End-organ perfusion refers a degree to which all organs of the patient are correctly perfused with blood. Every organ in a subject needs oxygen and nutrients to function. The perfusion is the process during which the blood enters the organ's tissue to bring the oxygen and nutrients. End-organ perfusion indicator is a measurement made of a subject that is the end-organ perfusion or that is correlated to the end-organ perfusion. The end-organ perfusion indicator has a value that changes in proportion to changes in end-organ perfusion of the subject.

The end-organ perfusion indicator may be measured by any method known in the art. Examples include:
mean femoral arterial pressure (MAPfemoral) measurement: MAPfemoral is an average pressure in a subject's femoral artery during one cardiac cycle. It is typically measured using pressure catheter located in femoral artery. An exemplary method for determining MAPfemoral, and an explanation of its meaning is disclosed in the textbook Kaplan's Essential of Cardiac Anesthesia (Kaplan, J. A, Elsevier Health Sciences, 2017, pages 203-208).
mean arterial pressure measured in another artery (MAP) measurement: MAP is an average pressure in a subject's artery during one cardiac cycle. It is typically measured using pressure catheter located in the artery in question. An exemplary method for determining MAP in another artery, and an explanation of its meaning is disclosed in the textbook Kaplan's Essential of Cardiac Anesthesia (Kaplan, J. A, Elsevier Health Sciences, 2017, pages 203-208).
lactate level measurement: Lactate level is a concentration of lactate in the arterial blood of the subject. It is typically measured by withdrawing a sample of arterial blood from the subject, and then analysing the blood using a standard method such as a lactate analyser or an enzymatic method. An exemplary method for determining lactate levels in an artery is given in Karagiannis, M. H., Reniker, A., Kerl, M. & Mann, F. A. Lactate Measurement as an Indicator of Perfusion (2006).
Spatial temperature gradient: Spatial temperature gradient is a temperature difference between two different parts of the body of the subject. It is typically measured using two skin temperature sensors, each obtaining a measurement at a different location on the subject. One location may be ventral surface of the big toe and another location may be a tympanic membrane. Alternatively, one location may be a fingertip and another location may be a forearm. An exemplary method for determining spatial temperature gradient is disclosed in Amson, H. et al. Core-to-skin temperature gradient measured by thermography predicts day-8 mortality in septic shock: A prospective observational study. J. Crit. Care 60, 294-299 (2020).
Peripheral perfusion index (PPI): The PPI is a ratio between pulsatile blood flow and non-pulsatile blood flow in the peripheral tissue. It is typically measured using a flow pulse oximeter located on the peripheral skin (e.g. finger) of the subject. An exemplary method for determining PPI is disclosed in Decreased peripheral perfusion measured by perfusion index is a novel indicator for cardiovascular death in patients with type 2 diabetes and established cardiovascular disease, Scientific Reports, https://www.nature.com/articles/s41598-021-81702-w.
Central venous-arterial CO₂ gap (P(v-a) CO₂): The P(v-a) CO₂ is the central-venous-arterial CO₂ gap that is a difference between CO₂ pressure measured in mixed venous blood (measured in the pulmonary arteries) and CO₂ pressure measured in arterial blood. It is typically measured using by obtaining a blood sample in each of a central vein and an artery, and measuring the dissolved oxygen content therein, using, for instance, a blood gas analyser. An exemplary method for determining P(v-a) CO₂ is disclosed in Mallat, J., Lemyze, M., Tronchon, L., Vallet, B. & Thevenin, D. Use of venous-to-arterial carbon dioxide tension difference to guide resuscitation therapy in septic shock. World J. Crit. Care Med. 5, 47-56 (2016).
Left ventricular strain (unitless): Left ventricular strain is a measure of deformation of the left ventricular (LV) wall that reflects LV function. It is typically measured using Doppler echocardiography (external). An exemplary method for determining left ventricular strain is disclosed in Støylen, A., Mølmen, H. E. & Dalen, H. Left ventricular global strains by linear measurements in three dimensions: interrelations and relations to age, gender and body size in the HUNT Study. Open Heart 6, e001050 (2019).

As mentioned, in response to the measured end-organ perfusion indicator being outside an accepted range, the target flow rate of the pump is adjusted by an increase.

To determine whether the end-organ perfusion indicator is outside an accepted range, the end-organ perfusion indicator is preferably monitored for a duration of an end-organ perfusion indicator sample period (EOPI_{sampleperiod}).

The EOPI_{sampleperiod} is typically between 1 and 10 seconds, preferably between 1 and 5 seconds. Consecutive EOPI_{sampleperiods} preferably run successively, i.e. at the end of a first EOPI_{sampleperiod}, a second EOPI_{sampleperiod} starts. Within a session, the duration of each EOPI_{sampleperiod} is preferably the same.

A value of end organ perfusion indicator is preferably determined for the EOPI_{sampleperiod}. The end organ perfusion indicator for the EOPI_{sampleperiod} is typically a statistical indicator for end organ perfusion indicator values acquired during the EOPI_{sampleperiod} such as, for instance, an order statistic (1st quartile, median, 3rd quartile) or an average (algebric, geometric). Preferably it is a median.

If value of end organ perfusion indicator determined for the EOPI_{sampleperiod} is outside the accepted range, the target flow rate of the pump is adjusted by an increase.

The accepted range is typically greater 50 to 70 mmHg, preferably 55 to 65 mmHg, when the measurement method is MAPfemoral. Based on the accepted range for MAPfemoral as a reference, an accepted range (and outside an accepted range) for other measurement methods may be determined. Exemplary accepted ranges and outside an accepted range for different end organ perfusion indicators are shown in Table 1 below.

TABLE 1
End organ perfusion indicators and accepted ranges.

| | | Outside |
|---|---|---|
| End-organ perfusion indicator | Accepted range | accepted range |
| Mean arterial pressure | ≥60 mmHg | <60 mmHg |
| Lactate level | ≤1.3 mmol/L | >1.3 mmol/L |
| Spatial temperature gradient: | ≤7° C. | >7° C. |
| Central to toe | | |
| Spatial temperature gradient:\| | ≤2° C. | >2° C. |
| fingertip to forearm | | |
| Peripheral perfusion index | ≤20% | >2 % |
| Central venous-arterial CO2 gap | ≤6 mmHg | >6 mmHg |
| Left ventricular longitudinal strain: | ≤−17% | >−17% |

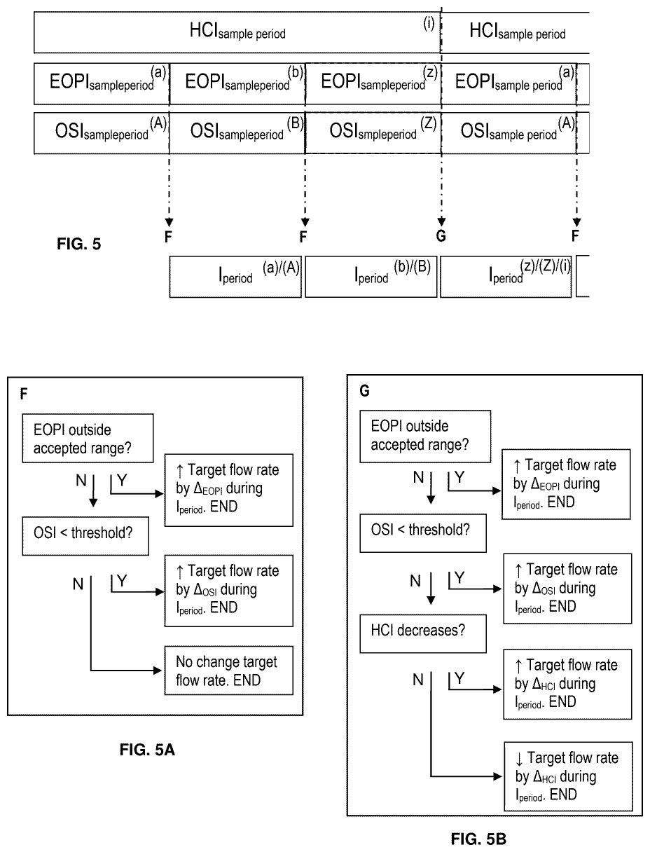

If the measured end-organ perfusion indicator is not outside the accepted range, the target flow rate of the pump is not increased responsive to the end-organ perfusion indicator. The flow rate may stay the same. However, it may still be adjusted responsive to the one of the other measured parameters such as heart contractility indicator and/or oxygen saturation indicator (see, for instance, FIG. 5).

The increase in target flow rate is implemented during an implementation period (I_{period}). During the implementation period (I_{period}), the existing target flow rate is gradually increased. The implementation period (I_{period}) is typically between 1 to 10 seconds, preferably between 1 to 5 seconds.

The increase in target flow rate during the implementation period (I_{period}) is typically 0.005-0.05 L/min, more preferably 0.015-0.025 L/min. The change in target flow rate due to end-organ perfusion indicator may be termed Δ_{EOPI}.

Once the implementation period (I_{period}) has expired, the target flow rate is maintained at the same level as at the end of the implementation period (I_{period}).

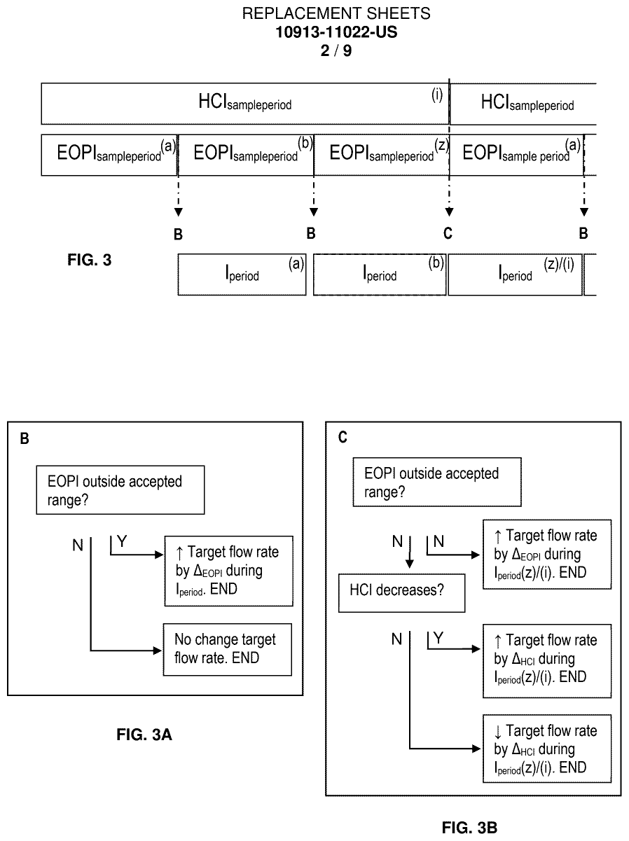

Preferably the multiple EOPI_{sampleperiods} within a session run consecutively. For instance, after the end of an EOPI_{sampleperiods}, a subsequent EOPI_{sampleperiods} starts. Consecutive EOPI_{sampleperiods} ((a), (b), (z)) are illustrated in FIG. 3. Based on the value of EOPI of an EOPI_{sampleperiods} at each point “B” (FIG. 3) with respect to an accepted range (FIG. 3A), the target flow is increased or not changed.

Where the set of measured parameters comprises the heart contractility indicator and end organ perfusion indicator, preferably the consecutive HCI_{sampleperiods} and consecutive EOPI_{sampleperiods} within a session are concurrent and synchronised. For instance, the beginning of each HCI_{sampleperiod} and EOPI_{sampleperiod} start at the same time. Within a duration of a HCI_{sampleperiod}, there are preferably multiple EOPI sampleperiods. There may be more than 10 (e.g. 20 to 600) EOPI_{sampleperiods} running consecutively with the duration of the HCI_{sampleperiod}. The end of the final EOPI_{sampleperiod} within the HCI_{sampleperiod} preferably ends at the same time as the end of the HCI_{sampleperiod}.

An exemplary synchronisation of consecutive HCI_{sampleperiods} and consecutive EOPI sampleperiods is depicted in FIG. 3. Based on the value of EOPI of an EOPI_{sampleperiod} at each point “B” with respect to an accepted range (FIG. 3A), the target flow is increased or not changed. At the end of each HCI_{sampleperiod}, which coincides with an and of an EOPI_{sampleperiod} (point “C” (FIG. 3)), if the value of EOPI is outside the accepted range, the target flow rate is increased by Δ_{EOPI} during the period (FIG. 3B). If the value of EOPI is within the accepted range, target flow rate is increased or decrease by Δ_{HCI} during the I_{period} depending on whether HCIs of a HCI_{sampleperiod} pair showed a decrease (FIG. 3B). Each point “B” is where each EOPI_{sampleperiod} ends, but does not coincide with an end of the HCI_{sampleperiod}. Each point “C” is where an EOPI sampleperiod and each HCI_{sampleperiods} ends at the same time.

Preferably, at the end of EOPI_{sampleperiod} an adjustment is made to the target flow rate. The adjustment is implemented during the implementation period (I_{period}). The I_{period} preferably runs subsequent to the previous EOPI_{sampleperiod}, and runs concurrently with the subsequent EOPI_{sampleperiod}. For instance, the beginning of I_{period} may start after the end of the previous EOPI sampleperiod. For instance, the beginning of I_{period} may start at the same time as the beginning of the subsequent EOPI_{sampleperiod}. A duration of each I_{period} may be the same (e.g. between 1 and 10 seconds, preferably between 1 and 5 seconds). Preferably, a duration of each I_{period} less than or equal to a duration of the EOPI_{sampleperiod}. Consecutive I_{periods} ((a), (b), (z)) are illustrated in FIG. 3; they start at the end of the previous EOPI_{sampleperiod}.

Where the set of measured parameters comprises the heart contractility indicator and end organ perfusion indicator, at the end of EOPI_{sampleperiod} and/or HCI_{sampleperiod}, an adjustment is made to the target flow rate. The adjustment is implemented during the implementation period (I_{period}). The I_{period} preferably runs subsequent to the previous EOPI_{sampleperiod} and/or HCI_{sampleperiod}, and runs concurrently with the subsequent EOPI_{sampleperiod}, or HCI_{sampleperiod}. In FIG. 3, the end of the previous HCI_{sampleperiod} (i) and EOPI_{sampleperiod} (z) is followed by I_{period} ((z)/(i)) which runs concurrently with the subsequent EOPI sampleperiod (a) and HCI_{sampleperiod}.

For instance, the beginning of I_{period} may start after the end of the previous EOPI_{sampleperiod} and/or HCI_{sampleperiod}. For instance, the beginning of I_{period} may start at the same time as the beginning of the subsequent EOPI_{sampleperiod} and/or HCI_{sampleperiod}. A duration of each I_{period} may be the same (e.g. between 1 and 10 seconds, preferably between 1 and 5 seconds). Preferably, a duration of each I_{period} less than or equal to a duration of the EOPI sampleperiod.

End organ perfusion indicator may be measured at intervals during the EOPI_{sampleperiod} e.g. every 0.1 to 2 seconds.

Each set of the multiple sets of measured parameters may further comprise an oxygen saturation indicator, wherein the target flow rate of the pump is further adjusted by an increase when the measured oxygen saturation indicator is less than a threshold value.

By monitoring and reacting to oxygen saturation indicator, the present method prevents or reduces organ damage caused by too low oxygen saturation (end organ ischemia). The method maintains the adjustment based on heart contractility within safety limits i.e. above the threshold value. By combining the monitoring of and reacting to oxygen saturation indicator with monitoring of and reacting to heart contractility indicator, the patient is slowly weaned off the VA-ECMO during the session, while reducing risk of end organ ischemia and Harlequin syndrome.

Oxygen saturation is a measure of the quantity of oxygen present in the blood of the subject. When the oxygen supply is insufficient to meet the metabolic demands of the tissues, an abnormal oxygen saturation indicator ensues and reflects an inadequacy in the systemic oxygenation. Oxygen saturation is, therefore, dependent on oxygen delivery and oxygen extraction.

Oxygen saturation indicator is a measurement made of a subject that is the oxygen saturation or that is correlated to oxygen saturation of the blood of the subject. The oxygen saturation indicator has a value that changes in proportion to changes in oxygen saturation of the subject.

The organ perfusion indicator may be measured by any method known in the art. Examples include:
Venous oxygen saturation (SvO₂): a measure of the oxygen content of the blood returning to the right side of the heart after perfusing the entire body. It is typically measured using a Swan-Ganz catheter placed in the pulmonary artery. An exemplary method for determining venous oxygen saturation is disclosed in Chetana Shanmukhappa, S. & Lokeshwaran, S. Venous Oxygen Saturation, in StatPearls (StatPearls Publishing, 2022).
Percutaneous oxygen saturation (SpO₂): a measure of peripheral arterial oxygen saturation. It is typically measured using a pulse oximeter located on the peripheral skin of the subject, typically on a finger of the subject. An exemplary method for determining percutaneous oxygen saturation is disclosed in Hafen, B. B. & Sharma, S. Oxygen Saturation, in StatPearls (StatPearls Publishing, 2022).
Oxygen reserve index (ORI): ORI is an oxygenation index that uses a multi-wavelength pulse oximeter. With this method, the sensor is able to evaluate arterial and veinous blood oxygen saturation. It is typically measured using a pulse oximeter located on the peripheral skin of the subject, typically on a finger of the subject. An exemplary method for determining ORI is disclosed in Ishida, Y., Okada, T., Kobayashi, T. & Uchino, H. ORiTM: a new indicator of oxygenation. J. Anesth. 35, 734-740 (2021).

Blood gas analysis: measurement of oxygen concentration in arterial blood from a blood sample. It is typically measured using by obtaining an arterial blood sample, and measuring the dissolved oxygen content therein, using, for instance, a blood gas analyser. An exemplary methods for performing blood gas analysis are disclosed in Arterial blood sampling. WHO Guidelines on Drawing Blood: Best Practices in Phlebotomy (World Health Organization, 2010) (how to sample arterial blood for blood gas analysis), and Williams, A. J. Assessing and interpreting arterial blood gases and acid-base balance. BMJ 317, 1213-1216 (1998) (working principle of oxygenation evaluation with a blood gas machine).

Preferably, oxygen saturation indicator is measured on the upper limbs and/or of the head.

As mentioned, in response to the measured oxygen saturation indicator being less than a threshold value, the target flow rate of the pump is further adjusted by an increase.

To determine whether the oxygen saturation indicator is less than the threshold value, the oxygen saturation indicator is monitored, and if its value is consecutively less than the threshold value for a duration of an oxygen saturation indicator sample period (OSI_{sampleperiod}), the oxygen saturation indicator is less than the threshold value.

The OSI_{sampleperiod} is typically between 1 and 10 seconds, preferably between 1 and 5 seconds. Consecutive OSI_{sampleperiods} preferably run successively, i.e. at the end of a first OSI_{sampleperiod}, a second OSI_{sampleperiod} starts. Within a session, the duration of each OSI_{sampleperiod} is preferably the same.

A value of oxygen saturation indicator is preferably determined for the OSI_{sampleperiod}. The oxygen saturation indicator for the OSI_{sampleperiod} is typically a statistical indicator for oxygen saturation indicator values acquired during the OSI_{sampleperiod} such as, for instance, an order statistic (1st quartile, median, 3rd quartile) or an average (algebric, geometric). Preferably it is a median.

The threshold value is typically 40 to 60%, preferably 45 to 55%, when the measurement method is venous oxygen saturation. Based on the threshold value for venous oxygen saturation as a reference, threshold value for other measurement methods may be determined. Exemplary threshold values, accepted and not accepted ranges for different oxygenation saturation indicator are shown in Table 2 below.

TABLE 2
Oxygen saturation indicators, threshold
values, accepted and not accepted ranges.

| | Threshold | Threshold |
|---|---|---|
| Oxygenation | value and | value and not |
| saturation indicator | accepted range | accepted range |
| Venous oxygen saturation | ≥50% | <50% |
| Percutaneous oxygen saturation | ≥90% | <90% |
| Oxygen reserve index | ≥0.25 | <0.25% |
| Blood gas analysis: SaO2 | ≥94% | <94% |

If the measured oxygen saturation indicator is not less than the threshold value, the target flow rate of the pump is not increased responsive to the oxygen saturation indicator. The flow rate may stay the same. However, it may still be adjusted responsive to the one of the other measured parameters such as heart contractility indicator and/or end organ perfusion indicator.

The increase in target flow rate is implemented during an implementation period (I_{period}). During the implementation period (I_{period}), the existing target flow rate is gradually increased. The implementation period (I_{period}) is typically between 1 to 10 seconds, preferably between 1 to 5 seconds.

The increase in target flow rate during the implementation period (I_{period}) is typically 0.005-0.05 L/min, more preferably 0.015-0.025 L/min. The change in target flow rate due to oxygen saturation indicator may be termed Δ_{OSI}.

Once the implementation period (I_{period}) has expired, the target flow rate is maintained at the same level as at the end of the implementation period (I_{period}).

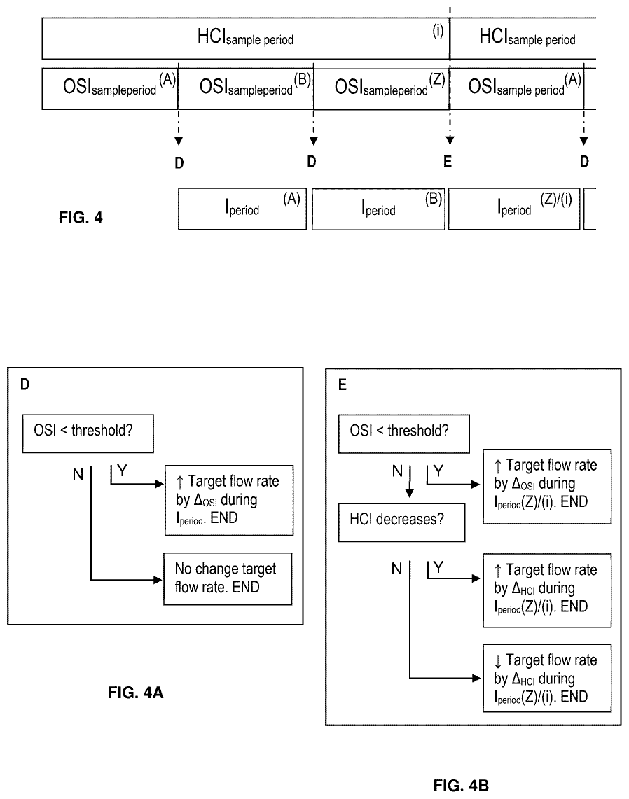

Preferably the multiple OSI_{sampleperiods} within a session run consecutively. For instance, after the end of an OSI_{sampleperiod}, a subsequent OSI_{sampleperiod} starts. Consecutive OSI_{sampleperiods} ((A), (B), (Z)) are illustrated in FIG. 4. Based on the value of OSI of an OSI_{sampleperiods} at each point “D” (FIG. 4) with respect to a threshold value (FIG. 4A), the target flow is increased or not changed.

Where the set of measured parameters comprises the heart contractility indicator and oxygen saturation indicator, preferably the consecutive HCI_{sampleperiods} and consecutive OSI_{sampleperiods} within a session are concurrent and synchronised. For instance, the beginning of each HCI_{sampleperiod} and OSI_{sampleperiod} start at the same time. Within a duration of a HCI_{sampleperiod}, there are preferably multiple OSI_{sampleperiods}. There may be more than 10 (e.g. 20 to 600) OSI_{sampleperiods} running consecutively with the duration of the HCI_{sampleperiod}. The end of the final OSI_{sampleperiod} within the HCI_{sampleperiod} preferably ends at the same time as the end of the HCI_{sampleperiod}.

An exemplary synchronisation of consecutive HCI_{sampleperiods} and consecutive OSI_{sampleperiods} is depicted in FIG. 4. Based on the value of OSI of an OSI_{sampleperiod} at each point “D” with respect to the threshold (FIG. 4A), the target flow is increased or not changed. At the end of each HCI_{sampleperiod}, which coincides with an and of an OSI_{sampleperiod} (point “E” (FIG. 4), if the value of OSI is outside the accepted range, the target flow rate is increased by Δ_{OSI} during the I_{period} (FIG. 4B). If the value of OSI is within the accepted range, target flow rate is increased or decrease by Δ_{HCI} during the I_{period} depending on whether HCIs of an HCI_{sampleperiod} pair showed a decrease (FIG. 4B). Each point “D” is where each OSI_{sampleperiod} ends, but does not coincide with an end of the HCI_{sampleperiod}. Each point “E” is where an OSI_{sampleperiod} and each HCI_{sampleperiods} ends at the same time.

Preferably, at the end of OSI_{sampleperiod} an adjustment is made to the target flow rate. The adjustment is implemented during the implementation period (I_{period}). The I_{period} preferably runs subsequent to the previous OSI_{sampleperiod}, and runs concurrently with the subsequent OSI_{sampleperiod}. For instance, the beginning of I_{period} may start after the end of the previous OSI_{sampleperiod}. For instance, the beginning of I_{period} may start at the same time as the beginning of the subsequent OSI_{sampleperiod}. A duration of each I_{period} may be the same (e.g. between 1 and 10 seconds, preferably between 1 and 5 seconds). Preferably, a duration of each I_{period} less than or equal to a duration of the OSI_{sampleperiod}. Consecutive I_{periods} ((A), (B), (Z/(i))) are illustrated in FIG. 4; they start at the end of the previous OSI_{sampleperiod} ((A), (B), (Z)).

Where the set of measured parameters comprises the heart contractility indicator and oxygen saturation indicator, at the end of OSI_{sampleperiod} and/or HCI_{sampleperiod}, an adjustment is made to the target flow rate. The adjustment is implemented during the implementation period (I_{period}). The I_{period} preferably runs subsequent to the previous OSI_{sampleperiod} and/or HCI_{sampleperiod}, and runs concurrently with the subsequent OSI_{sampleperiod}, or HCI_{sampleperiod}. In FIG. 4, the end of the previous HCI_{sampleperiod} (i) and OSI_{sampleperiod} (z) is followed by I_{period} ((Z)/(i)) which runs concurrently with the subsequent OS sampleperiod (A) and HCI_{sampleperiod}.

For instance, the beginning of I_{period} may start after the end of the previous OSI_{sampleperiod} and/or HCI_{sampleperiod}. For instance, the beginning of I_{period} may start at the same time as the beginning of the subsequent OSI_{sampleperiod} and/or HCI_{sampleperiod}. A duration of each I period may be the same (e.g. between 1 and 10 seconds, preferably between 1 and 5 seconds). Preferably, a duration of each I_{period} less than or equal to a duration of the OSI_{sampleperiod}.

Oxygen saturation indicator may be measured at intervals during the OS sampleperiod e.g. every 0.1 to 2 seconds.

Each set of the multiple sets of measured parameters may comprise a combination of the heart contractility indicator, the end-organ perfusion indicator, and the oxygen saturation indicator. If
the end-organ perfusion indicator is outside the accepted range, or
the oxygen saturation indicator is below the threshold value, or
there is a decrease in the heart contractility indicator,
   then
the target flow rate of the pump is adjusted by an increase;
   else
in response to a stable or increase in heart contractility indicator, the target flow rate is adjusted by a decrease

In more detail,
a) if the end-organ perfusion indicator is outside the accepted range,
then
   the target flow rate of the pump is adjusted by an increase, and there is a return to step a);
else
b) if the oxygen saturation indicator is below the threshold value,
then
   the target flow rate of the pump is adjusted by an increase, and there is a return to step a);
else
c) if there is a decrease in the heart contractility indicator,
   the target flow rate of the pump is adjusted by an increase, and there is a return to step a);
else
the target flow rate of the pump is adjusted by a decease, and there is a return to step a).

Preferably the consecutive HCI_{sampleperiods}, consecutive EOPI_{sampleperiods} and consecutive OSI_{sampleperiods} within a session are concurrent and synchronised. For instance, the beginning of each EOPI sampleperiod and OSI_{sampleperiod} start at the same time, and the end of each EOPI sampleperiod and OSI_{sampleperiod} end at the same time. Accordingly, a duration of each EOPI_{sampleperiod} and OSI_{sampleperiod} is the same (e.g. between 1 and 10 seconds, preferably between 1 and 5 seconds). An example of synchronised and concurrently running EOPI_{sampleperiods} and OSI_{sampleperiods} is shown in FIG. 5 (each EOPI_{sampleperiod} (a), (b), (z) and OSI_{sampleperiod} ((A), (B), (Z)) starts and stops at the same time).

Within a duration of a HCI_{sampleperiod}, there are preferably multiple EOPI_{sampleperiods} and OSI_{sampleperiods}. There may be more than 10 (e.g. 20 to 600) EOPI_{sampleperiods} running consecutively with the duration of the HCI_{sampleperiod}. Similarly, there may be more than 10 (e.g. 20 to 600) OSI_{sampleperiods} running consecutively with the duration of the HCI_{sampleperiod}. The multiple EOPI_{sampleperiods} and the multiple OSI_{sampleperiods} run concurrently. An example of multiple consecutive EOPI sampleperiods (a), (b), (z) and multiple consecutive OSI_{sampleperiods} (A), (B), (Z) running within a HCI_{sampleperiod} (i) is depicted in FIG. 5.

If the value of EOPI of an EOPI_{sampleperiod} at each point “F” was outside the accepted range (FIG. 5A), the target flow is increased by Δ_{EOPI} during the I_{period}. If the value of EOPI was not outside the accepted range, target flow rate is increased by Δ_{OSI} during the I_{period} if the value of OSI of an OSI_{sampleperiod} was less than the threshold (FIG. 5A). If both the value of EOPI was within the accepted range, and value of OSI was not less than the threshold, then there is no change in the target flow rate. Each point “F” is where each EOPI_{sampleperiod} and each OSI_{sampleperiod} ends at the same time, but does not coincide with an end of the HCI_{sampleperiod}.

If the value of EOPI of an EOPI_{sampleperiod} at each point “G” was outside the accepted range (FIG. 5A), the target flow is increased by Δ_{EOPI} during the I_{period}. If the value of EOPI was not outside the accepted range, target flow rate is increased by Δ_{OSI} during the I_{period} if the value of OSI of an OSI_{sampleperiod} was less than the threshold (FIG. 5A). If both the value of EOPI was within the accepted range, and value of OSI was not less than the threshold, then either
the target flow rate is increased by Δ_{HCI} during the I_{period} if there has been a decrease in HCI for an HCI_{sampleperiod} pair, or
the target flow rate is decreased by Δ_{HCI} during the I_{period} if there has been no decrease in HCI for the HCI_{sampleperiod} pair,
Each point “G” is where an EOPI_{sampleperiod} and an OSI_{sampleperiod} and each HCI_{sampleperiods} ends at the same time.

Preferably, at the end of EOPI_{sampleperiod}, OSI_{sampleperiod} or HCI_{sampleperiod}, an adjustment is made to the target flow rate. The adjustment is implemented during the implementation period (I_{period}). The I_{period} preferably runs concurrently with and is synchronised with the subsequent EOPI sampleperiod, or OSI_{sampleperiod} or HCI_{sampleperiod}. For instance, the beginning of I_{period} may start at the same time as the beginning of the next EOPI_{sampleperiod}, and OSI_{sampleperiod} and HCI_{sampleperiod}. A duration of each I_{period} may be the same (e.g. between 1 and 10 seconds, preferably between 1 and 5 seconds). Preferably, a duration of each I_{period} less than or equal to a duration of the EOPI_{sampleperiod} and/or OSI_{sampleperiod}. Consecutive I_{periods} ((a)/(A), (b)/(B), ((z)/(Z)/(i)) are illustrated in FIG. 5; they start at the end of the previous EOPI_{sampleperiod} ((a), (b), (z) and OSI_{sampleperiod} (A), (B), (Z). In FIG. 5, the end of the previous HCI_{sampleperiod} (i) and EOPI_{sampleperiod} (z) and OSI_{sampleperiod} (Z) is followed by I_{period} ((z)/(Z)/(i)) which runs concurrently with the subsequent EOPI sampleperiod (a), OSI_{sampleperiod} (A) and HCI_{sampleperiod}.

In more detail,
a) if the end-organ perfusion indicator is outside the accepted range,
then
   the target flow rate of the pump is adjusted by an increase, and there is a return to step a);
else
b) if the oxygen saturation indicator is below the threshold value,
then
   the target flow rate of the pump is adjusted by an increase, and there is a return to step a);
else
c) if there is a decrease in the heart contractility indicator,
then
   the target flow rate of the pump is adjusted by an increase, and there is a return to step a);
else
the target flow rate of the pump is adjusted by a decease, and there is a return to step a);
wherein
   end-organ perfusion indicator of step a) is determined for a duration EOPI sampleperiod,
   the oxygen saturation indicator of step b) is determined for a duration OSI_{sampleperiod},
   there are multiple EOPI_{sampleperiod} running consecutively and there are multiple OSI_{sampleperiod} running consecutively, each EOPI sampleperiod and OSI_{sampleperiod} are of the same length, start at the same time and run concurrently,
   the heart contractility indicator of step c) is determined for a duration HCI_{sampleperiod},
   there are multiple HCI_{sampleperiods} in a session running consecutively,
   during each HCI_{sampleperiod}, the multiple consecutively-running EOPI_{sampleperiods} run concurrently with the HCI_{sampleperiod},
   during the HCI_{sampleperiod}, the multiple consecutively-running OSI_{sampleperiods} run concurrently with the HCI_{sampleperiod},
   the target flow rate increase of step a) is implemented for a duration I_{period}, which starts after each EOPI_{sampleperiod} and runs concurrently with a subsequent EOPI_{sampleperiod}, or the target flow rate increase of step b) is implemented for the duration I_{period}, which starts after each OSI_{sampleperiod}, and runs concurrently with a subsequent OSI_{sampleperiod}, or the target flow rate increase or decrease of step c) is implemented for the duration I_{period}, which starts after each HCI_{sampleperiod}, and runs concurrently with a part of the subsequent HCI_{sampleperiod},
   a duration of the I_{period} is the same or less than a duration of the EOPI_{sampleperiod} Or OSI_{sampleperiod}.

By combining the monitoring of and reacting to heart contractility indicator, with monitoring of and reacting to end-organ perfusion indicator, with monitoring of and reacting to oxygen saturation indicator the patient is slowly weaned off the VA-ECMO during the session, while reducing risk of end organ ischemia and developing Harlequin syndrome. By monitoring end-organ perfusion, it is ensured that the organs are receiving enough blood flow to be correctly perfused or that they are not showing signs of low organ perfusion. By monitoring the oxygenation saturation indicator, the upper limbs or the head are not showing oxygen levels that are too low and that would therefore put the patient at risk of brain hypoxia

The determined target flow rate of the pump may be outputted directly to the pump, or be outputted to a feedback loop controller, which in turn controls the pump using a closed feedback loop based on a measured flow rate of the pump.

The determined target flow rate of the pump may be received by a feedback loop controller wherein the feedback loop controller is configured to:
receive a measured flow rate of the pump, that is a measurement of flow rate induced by the pump,
output a flow rate setting for the pump such that the measured flow rate of the pump approaches the determined flow rate of the pump.

Examples of the flow meter include Ultrasonic, Coriolis, magnetic, vortex, differential pressure or thermal type. Examples of suitable commercially available flow meters include Transonic ME 9PXL ultrasonic sensor. The flow meter is typically located at the start of the arterial cannula of the VA-ECMO, downstream of the oxygenator.

The feedback loop controller may include a “proportional-integral regulator”, PI regulator, which is known in the art. The PI regulator determines the flow rate setting based on a combination of a moment-to-moment difference between the measured flow rate and the target flow rate (“proportional” component), and an integral over time of a difference between the measured flow rate and the target flow rate (“integral” component).

An example of a PI regulator algorithm is given below:

$$u [ n ] = {K}_{P} . e [ n \} + \frac{{K}_{I} . T}{2} ( e [ n ] + e [ n - 1 ] ) + i [ n - 1 ]$$

$$u [ n ] is the output of the controller ( dynamic flow rate setting ) at the iteration n$$

$$e [ n ] is the error at the iteration n$$

$${K}_{P} is the proportional term$$

$${K}_{I} is the integrative term$$

$$T is the sampling period ( time between iterations )$$

The feedback loop controller may include an “Anti-windup controller”, which is known in the art. The anti-windup controller avoids excessive overshooting of the target flow rate. This may be achieved by any method, for instance, by avoiding that the flow rate setting for the pump is set to maximum (saturation). Typically, the anti-windup controller avoids excessive overshooting of the target flow rate when the feedback loop controller incorporates the proportional-integral regulator.

To this anti-windup controller an output clamp was also added. This means that the flow rate range is limited between a maximal and minimal value decided by the user.

The method is typically automatic, meaning there is no intervention or monitoring by the practitioner.

The method described herein is a computer implemented method. The method is in vitro, more in particular ex vivo.

Further provided is a device (interface device) for interfacing to a system for providing veno-arterial extracorporeal membrane oxygenation VA-ECMO of a subject during a VA-ECMO session, wherein the device is configured to dynamically determine the target flow rate of a pump of the system for providing VA-ECMO according to a method described herein. The device for interfacing to the VA-ECMO system typically comprises circuitry configured to perform the method described herein. The flow rate of the pump is adjusted to the target flow rate using a device for interfacing with the VA-ECMO system, an interface device, configured to adjust the flow rate of the pump responsive to determined target flow rate. The skilled person would understand how to configure an interface device.

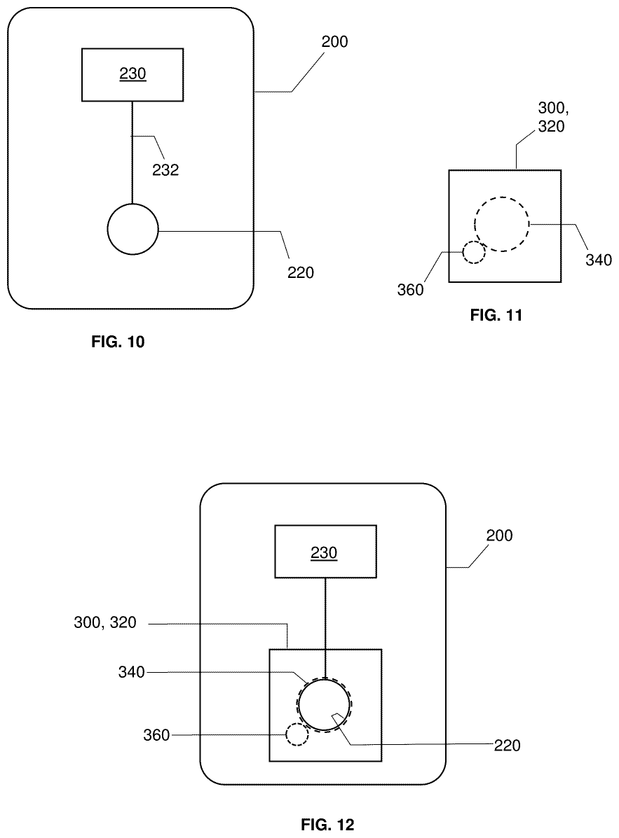

Shown in FIG. 10 is a basic illustration representative of an exterior of a VA-ECMO system (200) of the art highlighting a manually-actuated control dial (220) operatively linked (232) to a pump (230). Shown in FIG. 11 is a basic illustration of some components of an interface device (300) comprising a dismountable actuator assembly (320). Shown in FIG. 12 is a basic illustration of the interface device (300) interfaced to the manually-actuated control dial (220).

The interface device (300) may comprise a dismountable actuator assembly (320) for interfacing to the VA-ECMO system (200). The dismountable actuator assembly (320) may be configured:
to dismountably attach to the manually-actuated (rotary, linear) control dial (220) of the VA-ECMO system (200), wherein the manually-actuated control dial (220) adjusts the flow rate of the pump (230) of the VA-ECMO system (200);
to adjust by motorized actuation of the control dial (220) the flow rate of the pump (230) of the VA-ECMO system (200), responsive to the target flow rate of the pump determined by a method as described here.

The manually-actuated control dial (220) may or may not include a control knob that attaches to a moving shaft (rotating or sliding) of the VA-ECMO system (200) that causes a change in the flow rate of the pump. The moving shaft (rotating or sliding) may be a part of a potentiometer (rotary or linear) or a encoder (rotary or linear) or other electrical component capable of providing an indicator (e.g. an electronic signal, voltage amplitude) reflective of the position of the manually-actuated control dial (220). Typically, VA-ECMO system (200) is provided with a control knob that the practitioner grips to adjust the flow rate of the pump. The control knob may be dismountable thereby revealing the moving shaft. The dismountable actuator assembly (320) may be configured for dismountable attachment to the control knob or to the moving shaft.

The dismountable actuator assembly (320) preferably comprises a coupling element (340) configured for coupling to the manually-actuated control dial (220), such that a movement (e.g. rotating or sliding) of the coupling element (340) induces a corresponding movement of manually-actuated control dial (220). The coupling element (340) is configured for dismountable attachment to the manually-actuated control dial (220). The dismountable attachment of the coupling element (340) to the manually-actuated control dial (220) causes the coupling element (340) and the manually-actuated control dial (220) to move (e.g. rotating or sliding) in fixed relation to each other.

The dismountable actuator assembly (320) preferably includes a motor (360) (e.g. servo motor, stepper motor), configured to cause movement (e.g. rotating or sliding) of the manually-actuated control dial (220) or coupling element (340) responsive to the target flow rate of the pump (230) determined by a method as described here. A mechanical output of the motor is transmitted to the manually-actuated control dial (220) or coupling element (340).

The dismountable actuator assembly (320) may comprise a manual over-ride mechanism, configured for a manual over-ride of the motorized actuation of the control dial (220), allowing manual (mechanical) actuation of the control dial (20) via the dismountable actuator assembly (320). The manual over-ride may be implemented, for instance, by disengaging or depowering the motor (360).

The interface device (300) may comprise a computing device configured for performing a method as described herein. The computing device is preferably configured to control the motor (typically via a controller) responsive to the target flow rate of the pump determined by a method as described here. The computing device may be built into a housing of the interface device (300) or may be separate (e.g. running on a separate computing module). A housing of the interface device (300) may comprise one or more power and/or I/O ports (e.g. USB) for supply of power and/or exchange of data. Where the computing device is separate, the one or more power and/or I/O ports may be used conduct power and/or signals for control of movements of the motor.

The interface device may be at least partially, optionally fully comprised in an (pluggable) external module configured for control of the flow rate of the pump of the VA-EMCO system. The external module comprises a control signal output port for (electronic or optical) connection to a control signal input port of the VA-EMCO system. Signals outputted by the control signal output port control of the flow rate of the pump of the VA-EMCO system. The control signal output port may provide a serial output. Some VA-EMCO systems include a control signal input port (e.g. serial, USB, voltage) that allows connection to (plugging in of) an external module for control of the flow rate of a pump. A cable (e.g. USB, serial) typically connects the external module to the VA-EMCO system. The external module may comprise a computing device configured for performing a method as described herein. The computing device is preferably configured to generate signals for control of the pump responsive to the target flow rate of the pump determined by a method as described here. The computing device may be built into a housing of the external module or may be separate (e.g. running on a separate computing module). A housing of the external module may comprise one or more power and/or I/O ports (e.g. USB) for supply of power and/or exchange of data. Where the computing device is separate, the one or more power and/or I/O ports may be used conduct power and/or signals for control of movements of the motor.

The interface device may be at least partially, preferably fully comprised in a circuit board or other operative assembly of electronic components, for connection to existing circuitry of the VA-ECMO system, wherein the circuit board is configured for control of the flow rate of the pump thereof, and for performing the method as described herein. Such a circuit board may be retro-fitted to an existing VA-ECMO system. Such a circuit board may be configured for fitting within a housing of an existing VA-ECMO system. The circuit board may comprise a computing device configured for performing a method as described herein. The computing device is preferably configured to generate signals for control of the pump responsive to the target flow rate of the pump determined by a method as described here.

Other interface devices The above are non-limiting examples of an interface device. The skilled person would understand the components for and how to configure other interface devices according to known practices in the art.

Further provided is a system (a VA-ECMO system) for providing veno-arterial extracorporeal membrane oxygenation VA-ECMO to a subject during a VA-ECMO session, wherein the system is at least partially configured to dynamically determine the flow rate of a pump of the system according to a method described herein. The system comprises the VA-ECMO. The system typically comprises (existing or additional) circuitry configured to perform the method as described herein.

Further provided is a computing device (e.g. computing module, smart device, laptop) or system (e.g. VA-ECMO system) configured for performing a method as described herein. The computing device or system typically comprises circuitry configured to perform the method as described herein.

Typically circuitry comprises a processor and a memory. It may include one or more data input ports for receiving data (e.g. multiple sets of measured parameters) and/or one or more data input ports for sending data (output such as target flow rate).

Further provided is a computer program or computer program product having instructions which when executed by a computing device or system cause the computing device or system to perform a method as described herein.

Further provided is a computer readable medium having stored thereon a computer program (product) having instructions which when executed by a computing device or system cause the computing device or system to perform (each of the steps of) the method as described herein.

Further provided is a data stream which is representative of a computer program or computer program product having instructions which when executed by a computing device or system cause the computing device or system to perform (each of the steps of) the method as described herein.

The method, computing device, or system typically produces an output. The output may be a value (target flow rate), or a signal representative of the value. The output may be displayable or displayed on a display (e.g. computer screen, mobile device (smart phone tablet)). The output may be sent to the feedback loop controller where present.

The method may be performed using a standard computer system such as an Intel Architecture IA-32 based computer system 2, and implemented as programming instructions of one or more software modules stored on non-volatile (e.g. hard disk or solid-state drive) storage associated with the corresponding computer system. However, it will be apparent that at least some of the steps of any of the described processes could alternatively be implemented, either in part or in its entirety, as one or more dedicated hardware components, such as gate configuration data for one or more field programmable gate arrays (FPGAs), or as application-specific integrated circuits (ASICs), for example.

### Example 1

A subject (landrace swine) was provided with a Millar SPR-350S Mikro-Tip pressure catheter for measurement femoral arterial pressure; Femoral dP/dtₘₐₓ and MAP_{femoral} were determined from the signal. The subject was also provided with a Edwards Swan-Ganz catheter for measurement of venous oxygen saturation in the subject pulmonary artery; SvO₂ was determined from the signal. All three signals were recorded using a National Instrument DAQmx data acquisition system). The heart contractility indicator was dP/dtₘₐₓ, end-organ perfusion indicator was MAP_{femoral}, and the oxygen saturation indicator was SvO₂. Cardiogenic shock was induced in the animal by injection of total 4 mL of a microsphere solution over a period of approximately 30 minutes. After the injections, end-organ perfusion indicator (MAP_{femoral}) remained above its desired value, while continuous cardiac output (CCO) and oxygen saturation indicator (SvO₂) however, reached values lower than their respective thresholds. Shock was thus induced.

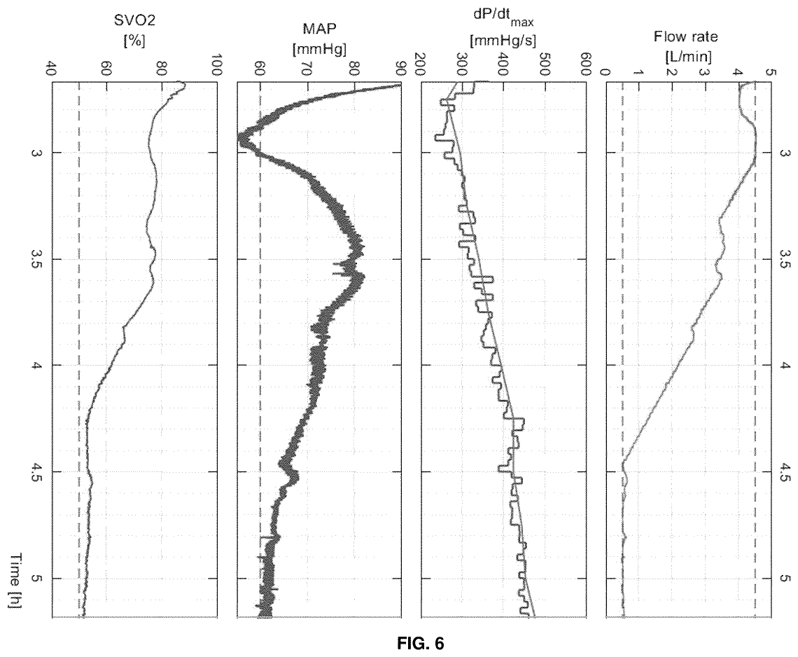

Full-flow VA-ECMO without the presently method was applied for an hour for stabilisation. After this period, the present method was used to control the VA-ECMO (FIGS. 6 to 8). The present method reduced flow of the VA-ECMO (FIG. 6), reacting to a rise in heart contractility indicator (dP/dtₘₐₓ) (2.5 to 4.5 hours), during which time the end-organ perfusion indicator (MAP_{femoral}) steadied above the threshold, and oxygen saturation indicator (SvO₂) steadied above the threshold. Hence, between 4.5 to 5 hours with a flow rate of ~0.5 L/min, the subject was effectively weaned off the VA-ECMO by the present method, allowing the heart and lungs to take over their natural function.

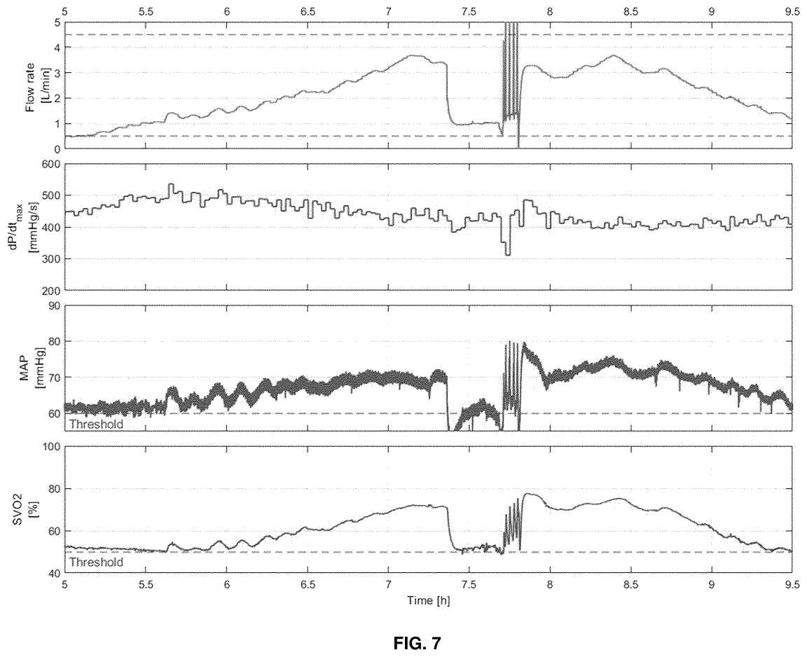

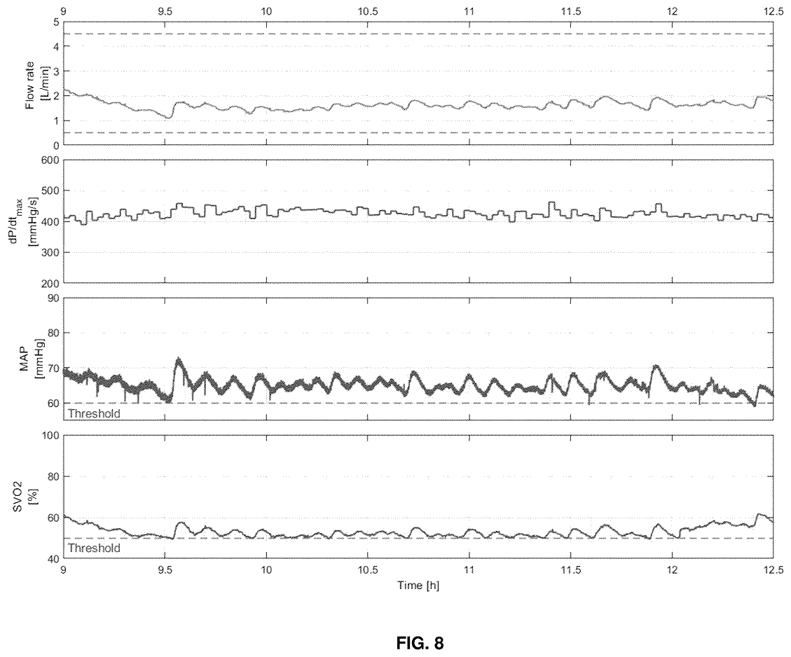

Measurements that continued until 12.5 hours after VA-ECMO using the present method (FIGS. 7 and 8) showed a response to changes in the subject requiring increased VA-ECMO support, with an increase in flow rate to 3.5 to 4 L/min (5 to ~8.5 hours) (FIG. 7). Between 7.5 and 8 h the flow of the VA-ECMO was changed to perform measurements. The flow rate was subsequently reduced using the present method and stabilised at 1 to 2 L/min (9.5 to 12.5 hours) which again allowed the heart and lungs to take over a substantial part of their natural function (FIG. 8).

### Example 2

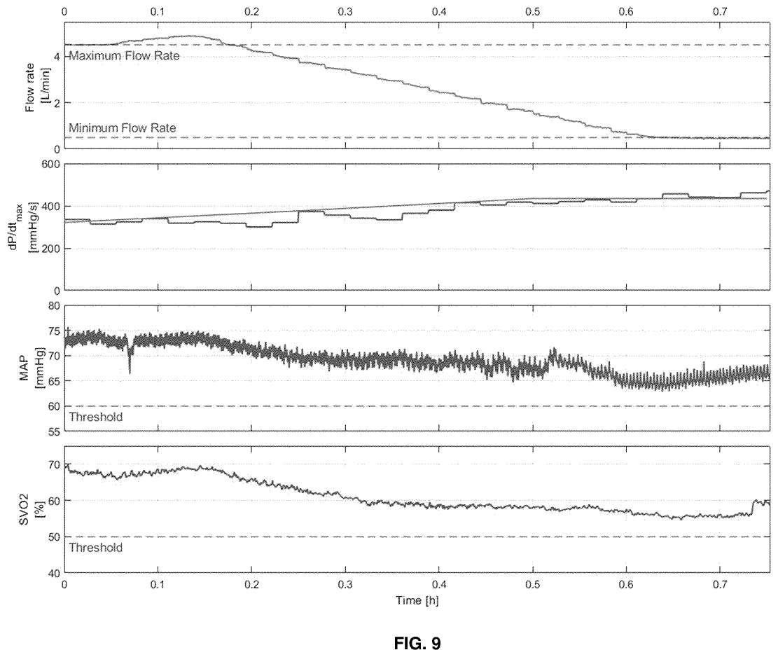

A VA-ECMO was cannulated on a healthy animal and heart contractility indicator (dP/dtₘₐₓ), end-organ perfusion indicator (MAP_{femoral}) and oxygen saturation indicator (SvO₂) were measured in conditions similar to Example 1. The method of the invention was applied to control the VA-ECMO, initially set to full flow. After about 30 minutes, the VA-ECMO fully weans itself (FIG. 9). It can be observed that the adaptive ECMO had no impact on the cardiac contractility as dP/dtₘₐₓ increases throughout the example.

1. A device for interfacing to a system for providing veno-arterial extracorporeal membrane oxygenation (VA-ECMO), a VA-ECMO system, to a subject during a VA-ECMO session, wherein the device is configured for dynamically determining a target flow rate of a pump of the VA-ECMO system using a method comprising:
receiving multiple sets of measured parameters (120) of the subject, wherein:
each set (120) of the multiple sets of measured parameters comprises a heart contractility indicator, and
the multiple sets of measured parameters are received over time during the VA-ECMO session,
determining (140), using the multiple sets of measured parameters, the target flow rate of the pump (160), and
adjusting the target flow rate of the pump, wherein
the target flow rate is adjusted by a reduction when heart contractility indicator is stable or increases, or
the target flow rate is adjusted by an increase when the heart contractility indicator decreases.

2. The device of claim 1,
wherein each set of the multiple sets of measured parameters further comprises an end-organ perfusion indicator, and
wherein the target flow rate of the pump is adjusted by an increase when the measured end-organ perfusion indicator is outside an accepted range.

3. The device of claim 1,
wherein each set of the multiple sets of measured parameters further comprises an oxygen saturation indicator, and
wherein the target flow rate of the pump is adjusted by an increase when the measured oxygen saturation indicator is less than a threshold value.

4. The device of claim 1,
wherein the target flow rate of the pump is received by a feedback loop controller
wherein the feedback loop controller is configured to:
receive a measured flow rate of the pump, that is a measurement of flow rate of the pump, and
output a flow rate setting for the pump such that the measured flow rate of the pump approaches the determined flow rate of the pump.

5. The device of claim 1, wherein each set of the multiple sets of measured parameters comprises:
the heart contractility indicator,
an end-organ perfusion indicator, and
an oxygen saturation indicator, and
wherein
the target flow rate of the pump is increased when
the oxygen perfusion indicator is below a threshold value, or
the venous oxygen saturation indicator is below a threshold value, or
there is a decrease in the heart contractility indicator.

6. The device of claim 1, wherein each set of the multiple sets of measured parameters comprises:
the heart contractility indicator,
an end-organ perfusion indicator, and
an oxygen saturation indicator, and
wherein
a) if the end-organ perfusion indicator is outside an accepted range,
then
the target flow rate of the pump is adjusted by an increase, and there is a return to step a);
else
b) if the oxygen saturation indicator is below a threshold value,
then
the target flow rate of the pump is adjusted by an increase, and there is a return to step a);
else
c) if there is a decrease in the heart contractility indicator,
then
the target flow rate of the pump is adjusted by an increase, and there is a return to step a);
else
the target flow rate of the pump is adjusted by a decease, and there is a return to step a);
wherein
end-organ perfusion indicator of step a) is determined for a duration EOPI_{sampleperiod},
the oxygen saturation indicator of step b) is determined for a duration OSI_{sampleperiod},
there are multiple EOPI_{sampleperiod} running consecutively and there are multiple OSI_{sampleperiod} running consecutively, each EOPI_{sampleperiod} and OSI_{sampleperiod} are of the same length, start at the same time and run concurrently,
the heart contractility indicator of step c) is determined for a duration HCI_{sampleperiod},
there are multiple HCI_{sampleperiods} in a session running consecutively,
during each HCI_{sampleperiod}, the multiple consecutively-running EOPI_{sampleperiods} run currently with the HCI_{sampleperiod},
during the HCI_{sampleperiod}, the multiple consecutively-running OSI_{sampleperiods} run currently with the HCI_{sampleperiod},
the target flow rate increase of step a) is implemented for a duration I_{period}, which starts after each EOPI_{sampleperiod} and runs concurrently with a subsequent EOPI_{sampleperiod}, or the target flow rate increase of step b) is implemented for the duration I_{period}, which starts after each OSI_{sampleperiod}, and runs concurrently with a subsequent OSI_{sampleperiod}, or the target flow rate increase or decrease of step c) is implemented for the duration I_{period}, which starts after each HCI_{sampleperiod}, and runs concurrently with a part of the subsequent HCI_{sampleperiod},
a duration of the I_{period} is the same or less than a duration of the EOPI_{sampleperiod} or OSI_{sampleperiod}.

7. The device of claim 1, further comprising a dismountable actuator assembly (320) for interfacing to the VA-ECMO system (200), the dismountable actuator assembly (320) configured to:
dismountably attach to a manually-actuated control dial (220) of the VA-ECMO system (200), wherein the manually-actuated control dial (220) adjusts the flow rate of the pump of the VA-ECMO system (200); and
adjust by motorized actuation of the control dial (220) the flow rate of the pump of the VA-ECMO system (200), responsive to the determined target flow rate of the pump.

8. The device of claim 1, that is at least partially comprised in an external module configured for control of the flow rate of the pump of the VA-EMCO system, wherein
the external module comprises a control signal output port for connection to a control signal input port of the VA-EMCO system, and
signals outputted by the control signal output port control of the flow rate of the pump of the VA-EMCO system.

9. The device of claim 1, comprised in a circuit board configured for electrical connection to existing circuitry of the VA-ECMO and for control of the flow rate of the pump thereof.

10. A system for providing veno-arterial extracorporeal membrane oxygenation (VA-ECMO) to a subject during a VA-ECMO session, wherein
the system is at least partially configured to dynamically determine the flow rate of a pump of the system according to a method comprising:
receiving multiple sets of measured parameters of the subject, wherein:
each set of the multiple sets of measured parameters comprises a heart contractility indicator, and
the multiple sets of measured parameters are received over time during the VA-ECMO session,
determining, using the multiple sets of measured parameters, the target flow rate of the pump, and
adjusting the target flow rate of the pump, wherein
the target flow rate is adjusted by a reduction when heart contractility indicator is stable or increases, or
the target flow rate is adjusted by an increase when the heart contractility indicator decrease.

11. A computer-implemented method for dynamically determining a target flow rate of a pump of a system for providing veno-arterial extracorporeal membrane oxygenation; (VA-ECMO) to a subject during a VA-ECMO session comprising:
receiving multiple sets of measured parameters of the subject, wherein:
each set of the multiple sets of measured parameters comprises heart contractility indicator; and
the multiple sets of measured parameters are received over time during the VA-ECMO session;
determining, using the multiple sets of measured parameters, the target flow rate of the pump, and
adjusting the target flow rate of the pump, wherein
the target flow rate is adjusted by a reduction when heart contractility indicator is stable or increases, or
the target flow rate is adjusted by an increase when the heart contractility indicator decreases.

12. The method according to claim 11,
wherein each set of the multiple sets of measured parameters further comprises an end-organ perfusion indicator, and
wherein the target flow rate of the pump is adjusted by an increase when the measured end-organ perfusion indicator is outside an accepted range.

13. The method according to claim 11,
wherein each set of the multiple sets of measured parameters further comprises an oxygen saturation indicator, and
wherein the target flow rate of the pump is adjusted by an increase when the measured oxygen saturation indicator is less than a threshold value.

14. The method according to claim 11, wherein the target flow rate of the pump is received by a feedback loop controller wherein the feedback loop controller is configured to:
receive a measured flow rate of the pump, that is a measurement of flow rate of the pump, and
output a flow rate setting for the pump such that the measured flow rate of the pump approaches the determined flow rate of the pump.

15. The method according to claim 11, wherein each set of the multiple sets of measured parameters comprises:
the heart contractility indicator,
an end-organ perfusion indicator, and
an oxygen saturation indicator,
wherein the method further comprises
increasing the target flow rate of the pump when
the oxygen perfusion indicator is below a threshold value, or
the venous oxygen saturation indicator is below a threshold value, or
there is a decrease in the heart contractility indicator.

16. The method according to claim 11, wherein each set of the multiple sets of measured parameters comprises:
the heart contractility indicator,
an end-organ perfusion indicator, and
an oxygen saturation indicator,
wherein
a) if the end-organ perfusion indicator is outside an accepted range,
then
the target flow rate of the pump is adjusted by an increase, and there is a return to step a);
else
b) if the oxygen saturation indicator is below a threshold value,
then
the target flow rate of the pump is adjusted by an increase, and there is a return to step a);
else
c) if there is a decrease in the heart contractility indicator,
then
the target flow rate of the pump is adjusted by an increase, and there is a return to step a);
else
the target flow rate of the pump is adjusted by a decease, and there is a return to step a);
wherein
end-organ perfusion indicator of step a) is determined for a duration EOPI_{sampleperiod},
the oxygen saturation indicator of step b) is determined for a duration OSI_{sampleperiod},
there are multiple EOPI_{sampleperiod} running consecutively and there are multiple OSI_{sampleperiod} running consecutively, each EOPI_{sampleperiod} and OSI_{sampleperiod} are of the same length, start at the same time and run concurrently,
the heart contractility indicator of step c) is determined for a duration HCI_{sampleperiod},
there are multiple HCI_{sampleperiods} in a session running consecutively,
during each HCI_{sampleperiod}, the multiple consecutively-running EOPI_{sampleperiods} run currently with the HCI_{sampleperiod},
during the HCI_{sampleperiod}, the multiple consecutively-running OSI_{sampleperiods} run currently with the HCI_{sampleperiod},
the target flow rate increase of step a) is implemented for a duration I_{period}, which starts after each EOPI_{sampleperiod} and runs concurrently with a subsequent EOPI_{sampleperiod}, or the target flow rate increase of step b) is implemented for the duration I_{period}, which starts after each OSI_{sampleperiod}, and runs concurrently with a subsequent OSI_{sampleperiod}, or the target flow rate increase or decrease of step c) is implemented for the duration I_{period}, which starts after each HCI_{sampleperiod}, and runs concurrently with a part of the subsequent HCI_{sampleperiod},
a duration of the I_{period} is the same or less than a duration of the EOPI_{sampleperiod} or OSI_{sampleperiod}.

17. The method according to claim 11, wherein the flow rate of the pump is adjusted to the target flow rate using a device for interfacing with the VA-ECMO system, an interface device, configured to adjust the flow rate of the pump responsive to determined target flow rate.

18. The method according to claim 17, wherein the interface device comprises a dismountable actuator assembly for interfacing to the VA-ECMO system, the dismountable actuator assembly (320) configured to:
dismountably attach to a manually-actuated control dial of the VA-ECMO system, wherein the manually-actuated control dial adjusts the flow rate of the pump of the VA-ECMO system; and
adjust by motorized actuation of the control dial the flow rate of the pump of the VA-ECMO system, responsive to the determined target flow rate of the pump.

19. The method according to claim 17, wherein the interface device is at least partially comprised in an external module provided with a control signal output port, configured for control of the flow rate of the pump of the VA-EMCO system disposed with a control signal input port.

20. The method according to claim 17, wherein the interface device is comprised in a circuit board configured for electrical connection to existing circuitry of the VA-ECMO and for control of the flow rate of the pump thereof, wherein the circuit board is configured to perform the method.

21. (canceled)

22. (canceled)

23. (canceled)

24. (canceled)
